(19) United States
(12) Patent Application Publication
(10) Pub. No.: US 2005/0195155 A1
(43) Pub. Date: Sep. 8, 2005

(54) Method and device for providing input to a computer system via one or both of breathing and biting

(76) Inventors: Pierre Bonnat, Grieges (FR); Venkatraman Parameswaran, Las Vegas, NV (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/793,987

(22) Filed: Mar. 4, 2004

(51) Int. Cl. A61F 2/36

(52) U.S. Cl. 345/156

(57) ABSTRACT

A device to facilitate a user interface of a computer system utilizing one or both of fluid flow through the device, for example human breadth, and deformation of the device, for example by biting or chewing. The device includes a flexible body that defines a fluid current channel with an inlet and outlet. The flexible body includes at least a region of relatively lesser stiffness and at least a region of relatively greater stiffness. A displaceable member is attached to the flexible body at the region of greater stiffness. The displaceable member is capable of motion in response to fluid flow through the fluid current channel as well as to deformation of the flexible body. The device may include a sensor to react to a movement of the displaceable member and a processor to process the electrical signals from the sensor.

### FIELD OF THE INVENTION

The present invention concerns the technical field of providing input to a computer system.

### BACKGROUND

A control device known as a mouse is widely used for transforming movements into input and control operations required by a user of a computer system. The mouse typically comprises a housing equipped with electronic means connected to the computer for transforming the movements of the housing on the working surface into a movement of the cursor or pointer on the computer screen.

A mouse typically includes a ball for rolling over the working surface, sensors to detect the movements of the ball and a processor to process the electric signals of the sensors. The processor is connected to the computer by an electric cable or a Hertzien or infrared link. The processor delivers signals recognised by the protocol of the port to which the mouse is connected, usually corresponding to the standard RS 232. However, the mouse can also be connected to the computer via a dedicated interface card or to a specific bus in which case the processor delivers one or several signals recognised by the protocol associated with this interface card or bus.

The mouse may in addition include a certain number of push or scrolling buttons which are also connected to the processor and which correspond to validation or data entry function according to the operating mode of the computer.

The processor may provide several principal functions, namely:
detection of the movement of the mouse,
detection of the position of the push-buttons,
and communication with the computer as per the retained standard.

Communication with the microcomputer is usually managed by a microprocessor ensuring the two parts of the processing of the signals derived from the movement and position detectors of the push-buttons

Finally, the mouse is associated with control software loaded into the computer, which decodes the signal transmitted by the mouse. The driver provides a requesting application software with information concerning the state and status of the mouse: firstly the movement and secondly the position of push-buttons so as to enable the software application to carry out the resultant actions.

In its most frequently used operating mode, the driver communicates with the sub-programme or movement routine of the cursor or pointer when the mouse is moved and sends messages to the programme when the push-buttons of the mouse are pressed.

### SUMMARY OF THE INVENTION

According to various aspects of the present invention, a method of manufacture, method of use and device to provide input to a computer system are provided. The device includes a flexible body, and a processor which can transmit electronic signals in response to one or both of fluid flow through the device, for example human breath, and deformation of the device, for example by biting or chewing. Other features of the present invention will be apparent from the accompanying drawings and from the detailed description, which follows.

### BRIEF DESCRIPTION OF THE DRAWINGS

The present invention is illustrated by way of example and not limitation in the figures of the accompanying drawings, in which like references indicate similar elements and in which:

FIG. 1 shows a diagrammatic view of an exemplary embodiment of the input device connected to and able to provide input to a computer system.

FIG. 2 shows a diagrammatic view of one embodiment of the input device, wherein a rigid ring is embedded within a tube.

FIG. 3 shows a diagrammatic view of another embodiment of the input device with extensions into the fluid current channel.

FIG. 4 shows a diagrammatic view of the embodiment of the input device shown in FIG. 3 while under a deformation force, such as biting by one or both of lips and teeth.

FIG. 5 shows a diagrammatic view of yet another embodiment of the input device.

FIG. 6 shows a further diagrammatic view of the embodiment shown in FIG. 5 under the influence of a fluid flow from the inlet to the outlet of the input device.

FIG. 7 shows a diagrammatic view of yet another embodiment of the input device, which comprises multiple regions of relatively greater stiffness, and which is capable of providing multiple unique inputs.

FIG. 8 shows a partial diagrammatic view of yet another embodiment of the input device as well as the various input forces that could be used to provide input.

FIG. 9 shows a diagrammatic view of an alternate embodiment of the input device connected to and able to provide input to a computer system.

FIG. 10 shows a diagrammatic view of the alternate embodiment connected to the computer system in FIG. 9.

FIG. 11 shows yet another alternate embodiment of the input device.

FIG. 12 shows the embodiment shown in FIG. 10 under the influence of a deformation force, such as biting by one or both of lips and teeth.

FIG. 13 shows a process diagram describing a method of manufacture for the input device.

FIG. 14 shows a process diagram describing an alternate method of manufacture for the input device.

FIG. 15 shows a process diagram describing 3 methods of use for the input device, which can be employed independently or in combination.

FIG. 16 shows 3 embodiments of the input device wherein the device is embedded or attached to a headset or helmet.

FIG. 17 illustrates one example of an application of the input device wherein the cursor movement is controlled by the deflection of segments under the influence of a deformation force, such as biting by one or both of lips and teeth.

FIG. 18 shows the embodiment shown in FIG. 17 under the influence of a deformation force, such as an expiration flow or inspiration flow of fluid.

FIG. 19 shows yet another alternate embodiment of the input device in which a mobile element can come to push against the segments.

### DETAILED DESCRIPTION

A method and device for providing input to a computer system is described. In the following description, for purposes of explanation, numerous specific details are set forth in order to provide a thorough understanding of the present invention. It will be evident, however, to one skilled in the art that the present invention may be practiced without these specific details.

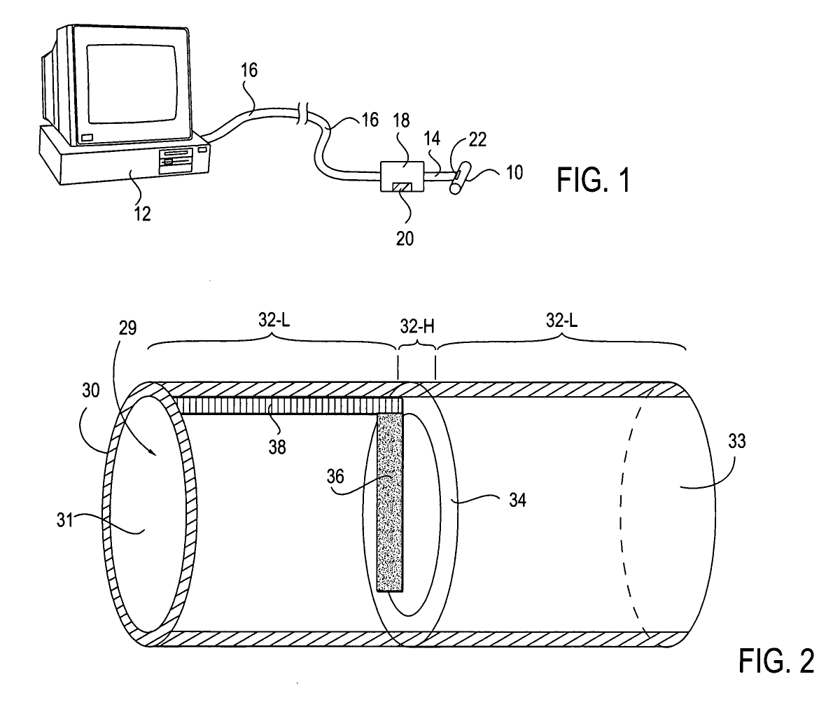

FIG. 1 illustrates an input device 10, according to an exemplary embodiment of the present invention, as it may be used in connection with a computer system 12. In this embodiment, a connector 14 connects the input device 10 to an interface box 18. In various embodiments, the connector 14, may be a link such as a networking cable or wireless connection such as BLUETOOTH, radio frequency, or infrared and hardware and software required by the box 18 and the input device 10 to communicate via the link. The interface box 18 contains a processor 20 to process the electric signal arriving from a converter 22 embedded within the input device 10. The box 18 is connected to a computer system 12 via a connector 16. The connector 16 may be a link such as a networking cable or a wireless connection such as BLUETOOTH, radio frequency, or infrared and any hardware and software required by the box 18 and the computer system 12 to communicate via the link. Alternatively, the input device 10 may be designed such that the box 18 and its components are embedded in the input device. The connector 14 is then not required, thereby simplifying the design.

In the exemplary embodiment shown in FIG. 1, a user can provide input to the computer system 12 to monitor, interact with, and control the computer system 12 by chewing, biting with lips or teeth, breathing into, sucking on, and/or inhaling through the input device 10.

FIG. 2 shows a cut-out view of one embodiment of the input device 10. In this embodiment, a flexible body 30, in the exemplary form of a tube, defines a fluid current channel 29 with an inlet 31 and an outlet 33. The flexible body 30 may be constructed of a flexible material such as rubber. The flexible body 30 is divided into at least one region of relatively lesser stiffness 32-L, and at least one region of relatively greater stiffness 32-H. In this embodiment, a ring 34 is located within the fluid current channel 29. The ring 34 may be inserted into the body 30 without permanently attaching to it so that the ring can be removed, maintained and replaced with ease. As a result of the placement of the ring 34, the region of the flexible body 30 around the ring has a relatively greater stiffness (or resistance to deformation) in comparison with the rest of the flexible body 30. It will be appreciated by one skilled in the art that in addition to inserting a structural element such as a ring, the region of relatively greater stiffness can be defined by many other techniques, for example, doping the material in different regions in order to obtain higher and lower levels of stiffness, or defining the flexible body to be thicker and thinner in regions of different stiffness.

FIG. 2 also shows a displaceable member in the exemplary form of a deflection segment 36, attached to the flexible body 30 at, or in proximity to, the region of greater stiffness 32-H. The deflection segment 36 is capable of motion in response to fluid flows through the fluid current channel 29 as well as to deformation of the flexible body 30. Deflection of this segment 36 can be detected by sensors and processed as described in U.S. Pat. No. 6,574,571, which is hereby incorporated by reference.

A lever segment 38 is attached to the region of greater stiffness 32-H. The lever segment 38 extends beyond the region of relatively greater stifffness 32-H into one of the regions of relatively lesser stiffness 32-L. When the flexible body 30 in the region of relatively lesser stiffness 32-L is deformed, as shown in FIG. 4, the lever segment 38 can cause the deflection segment 36 to be displaced.

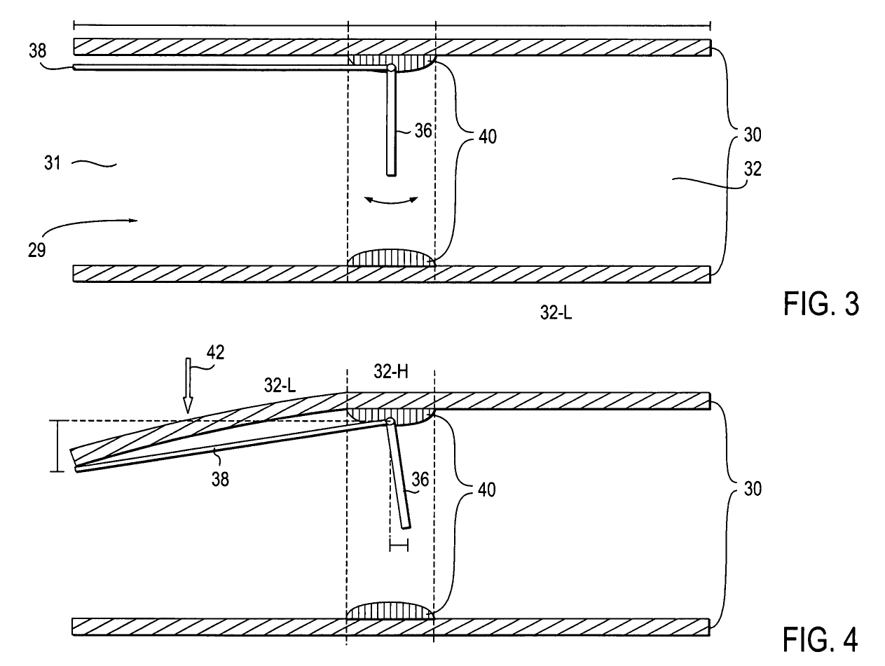

FIG. 3 shows a diagrammatic view of another embodiment of the input device 10. In this embodiment, the region of relatively greater stiffness 32-H is created by extensions 40 of the flexible body 30 into the fluid current channel 29. This view also shows that the deflection segment 36 can be capable of bidirectional displacement within the fluid current channel 29. Because bidirectional displacement is allowed, fluid currents entering and exiting the inlet 31 of the fluid current channel 29 can be used to provide input. For example, a user could breath into the input device 10 to provide one type of input, and inhale near the inlet 31 of the input device 10 in order to provide a second input. Additionally, a user could place their lips around the apparatus and suck in or blow out to provide more forceful current flow in either direction.

FIG. 4 shows an embodiment of the input device 10 where the region of relatively lesser stiffness 32-L is being deformed by a force 42. In one potential use of the input device 10, this force 42 can come from the user biting the region of relatively lesser stiffness 32-L with one or both of lips and teeth. As a result of the deformation of the flexible body 30 in the region of relatively lesser stiffness 32-L, the lever segment 38 can be displaced, thereby causing displacement of the deflection segment 36. The sensors used to sense displacement of the deflection segment 36 in response to a fluid flow as described in U.S. Pat. No. 6,574,571 can also be used to sense displacement of the deflection segment 36 in response to deformation of the region of relatively lesser stiffness 32-L.

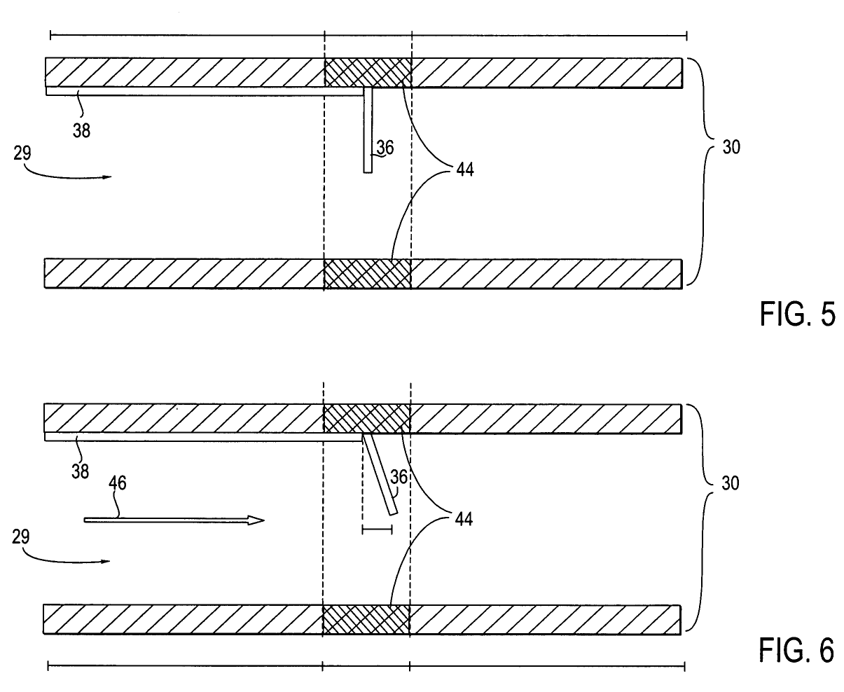

FIG. 5 displays a diagrammatic view of another embodiment of the input device 10. In this view, a region of relatively greater stiffness 44 is contained within the walls of the flexible body 30. It will be appreciated by one skilled in the art that it is possible to create this region by many methods, for example, doping the flexible body material in the region of relatively greater stiffness 44, or using a different material for the region of relatively greater stiffness 44 than the rest of the flexible body 30.

FIG. 6 displays an embodiment of the input device 10 subject to a fluid flow 46 through the fluid channel 29. As a result of the pressure from the fluid flow 46, the deflection segment 36 can be displaced in the direction of the fluid flow 46. This displacement can be sensed by the converter 22 and processed by the processor 20 via the sensors and methods described in U.S. Pat. No. 6,574,571.

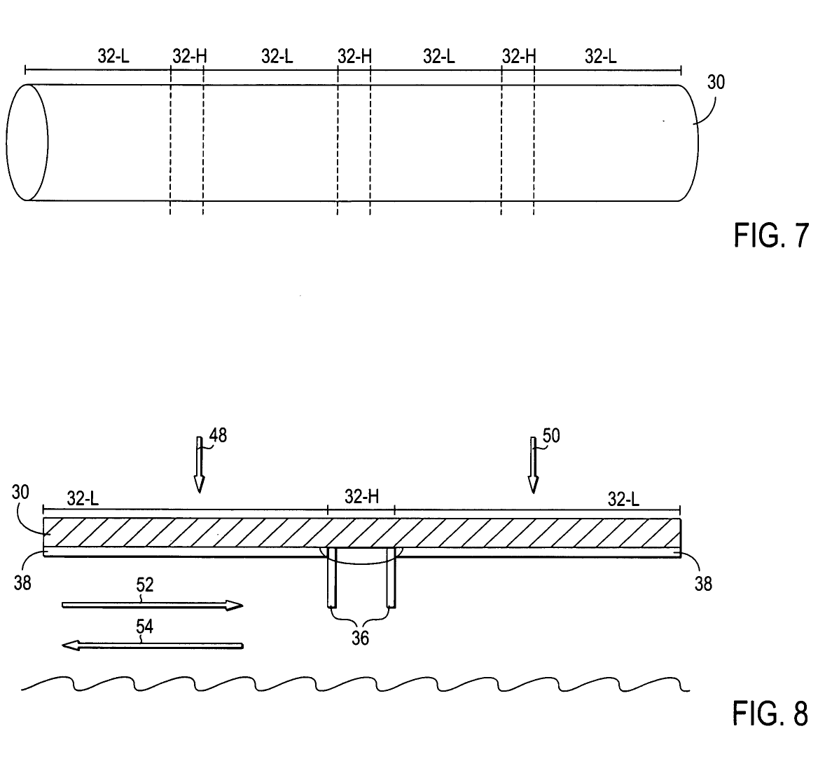

FIG. 7 shows an alternate embodiment of the input device 10 where multiple regions of relatively greater stiffness 32-H are alternated with regions of relatively lesser stiffness 32-L on the same flexible body 30. An advantage of this embodiment is that a user may bite or chew with the lips or teeth in several different regions of the device in order to create multiple input signals for providing input to a computer system 12.

FIG. 8 shows a partial view of another embodiment of the input device 10. In this embodiment, two deflection segments 36 and two lever segments 38 are attached to the same region of relatively greater stiffness 32-H. In this embodiment, two forces 48 and 50 can be applied to the flexible body 30, one in each region of relatively lesser stiffness 32-L. Since there are two of them, the deflection segments 36 do not need to be bi-directional in this embodiment. One deflection segment 36 can be configured to move in response to a fluid flow 52 entering the flexible body 30 from the inlet 31, while the other deflection segment 36 can be configured to move in response to a fluid flow 54 exiting the flexible body 30 through the inlet. In this embodiment, a user could bite or otherwise apply a force in two areas, and breath into and out of the inlet 31 in order to provide multiple inputs to the computer system 12.

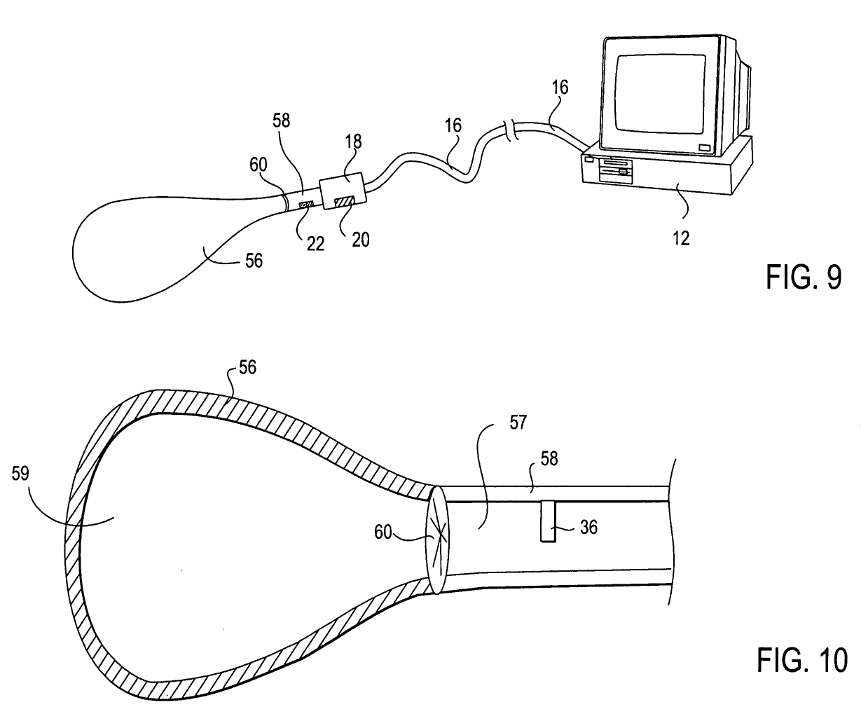

FIG. 9 shows another embodiment of the input device 10 in connection with a computer system 12. In this embodiment, a flexible body 56 defines a fluid chamber with a single opening. The opening is connected to a pressure valve 60. The pressure valve 60 is also connected to a second body 58 which defines a fluid current channel. A converter 22 is embedded within the fluid current channel and converts fluid flow through the body 58 that defines the fluid current channel into electrical signals. An interface box 18 containing a processor 20 to process the electrical signals from the converter 22 is connected to the fluid current channel. A connector 16, such as a networking cable or a wireless connection such as BLUETOOTH, radio frequency, or infrared and the necessary hardware and software, facilitates communication between the interface box 18 and the computer system 12.

FIG. 10 shows a magnified, cross-sectioned view of one embodiment of the input device 10. The flexible body 56 is connected to a body 58 defining a fluid current channel 57. The pressure valve 60 separates the body 58 defining the fluid current channel 57 from the fluid chamber 59 encompassed within the flexible body 56, which is filled with one or more fluids in one or both of gaseous and liquid states. The pressure valve 60 is capable of opening and closing in response to pressure changes within the fluid chamber 59 or the fluid current channel 57. For example, deformation of the flexible body 56 will result in increased pressure within the fluid chamber 59. In response, the pressure valve 60 can open and allow fluid to flow through the fluid current channel 57. A deflection segment 36 is pivotably mounted within the fluid current channel 57 and is capable of motion in response to fluid flows through the fluid current channel 57. The motion of the deflection segment 36 can be sensed and processed in the manner described in U.S. Pat. No. 6,574,571.

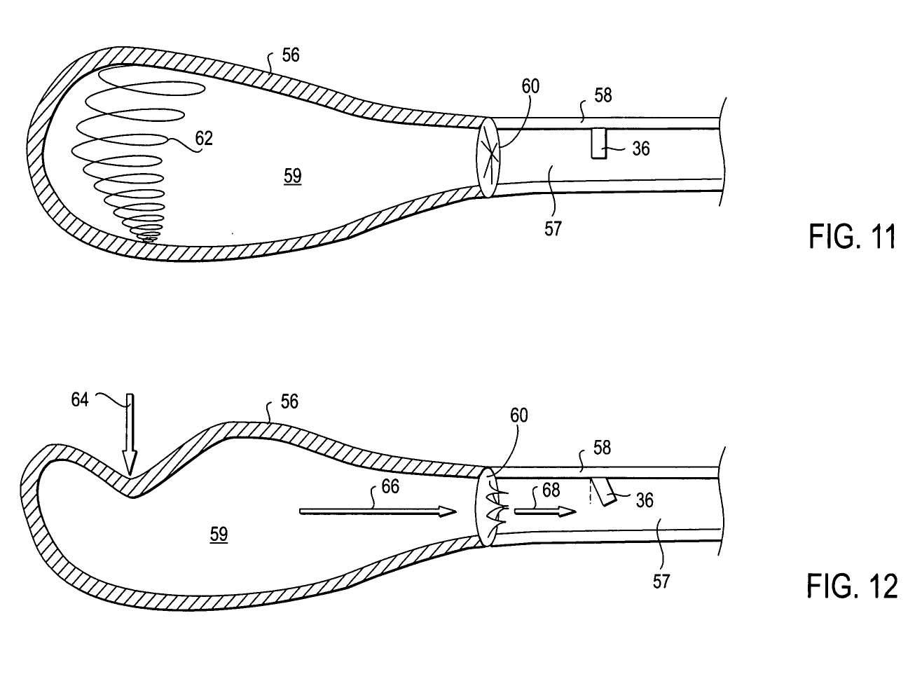

FIG. 11 shows an alternate embodiment of the invention where a spring 62 is placed within the fluid chamber 59 defined by the flexible body 56. After the flexible body 56 is deformed, the spring 62 can aid the flexible body 56 in returning to its original configuration. When the spring 62 forces the flexible body 56 to open after deformation, it can also create a drop in pressure within the fluid chamber 59. The pressure valve 60 can open in response to lower pressure within the fluid chamber 59 allowing fluid to return to the fluid chamber 59. The deflection segment 36 may be affixed to, or integrally formed with, the body 58 defining the fluid current chamber 58 such that it is able to deflect in response to fluid flows entering the fluid chamber 59, as well as in response to fluid flows exiting the fluid chamber 59. Additionally, the deflection segment 36 can be affixed in such a manner as to only allow one-way deflection.

FIG. 12 shows a diagram of an embodiment of the input device 10 in use. When a force 64, such as biting by one or both of the lips or teeth of a user, is applied, the flexible body 56 can deform and increase the pressure within the fluid chamber 59. In response to the increased pressure, the pressure valve 60 can open, allowing a fluid flow 66 out of the fluid chamber 59 and into the fluid current channel 58. The fluid flow 68 within the fluid current channel 58 can cause the deflection segment 36 to be displaced. The displacement of the deflection segment 36 can be sensed and processed in the manner described in U.S. Pat. No. 6,574,571.

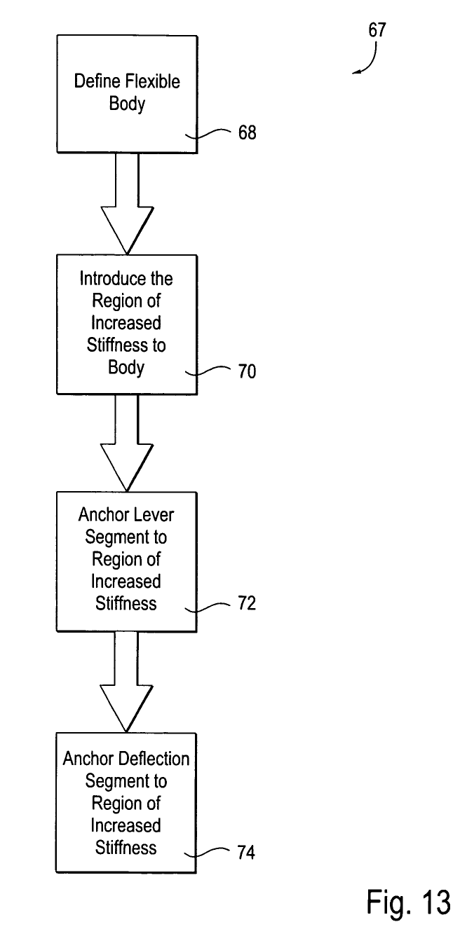

FIG. 13 shows a flow diagram for a method 67 of manufacturing the input device 10. A first operation 68 is to define a flexible body 30 that creates a fluid current channel having an inlet and an outlet. One skilled in the art will appreciate that there are several methods available to create a flexible body as defined, for example, injection molding or wax molding with a flexible material such as natural or synthetic rubber. The next operation 70 is to define at least one region of relatively greater stiffness 32-H within the flexible body. The region of relatively greater stiffness 32-H can be defined within the flexible body in a number of ways. For example, the region of relatively greater stiffness 32-H can be defined by using a material that is stiffer than the flexible material used in the region of relatively lesser stiffness 32-L or by altering the material of the flexible body chemically, with heat, or by working it in the region of greater stiffness. Alternatively, the region of relatively greater stiffness 32-H can be braced from within the flexible body by inserting a brace or frame within the fluid current channel.

The next operation 72 in the method of manufacturing the input device shown is to anchor the lever segment 38 to the region of greater stifffness 32-H within the flexible body. One skilled in the art will appreciate that anchoring the lever segment 38 can be achieved, for example, with a pin, a screw, or glue, or by the flexible body being manufactured such that there is an insertion for the lever segment 38.

The next operation 74 in the method 67 of manufacturing the input device is to anchor the deflection segment 36 to the region of greater stiffness 32-H. Anchoring the deflection segment 36 to the region of greater stiffness 32-H can be achieved by a similar variety of methods as described above for operation 72. One skilled in the art will appreciate that the lever segment 38 and the deflection segment 36 can be designed as a single body to be inserted into the flexible body. When the lever segment 38 and the deflection segment 36 are attached to the region of greater stiffness 32-H, it is done in such a way that in response to a force applied in the region of lesser stiffness 32-L, the lever segment 38 is able to move and is able to push the deflection segment 36, which is also able to move.

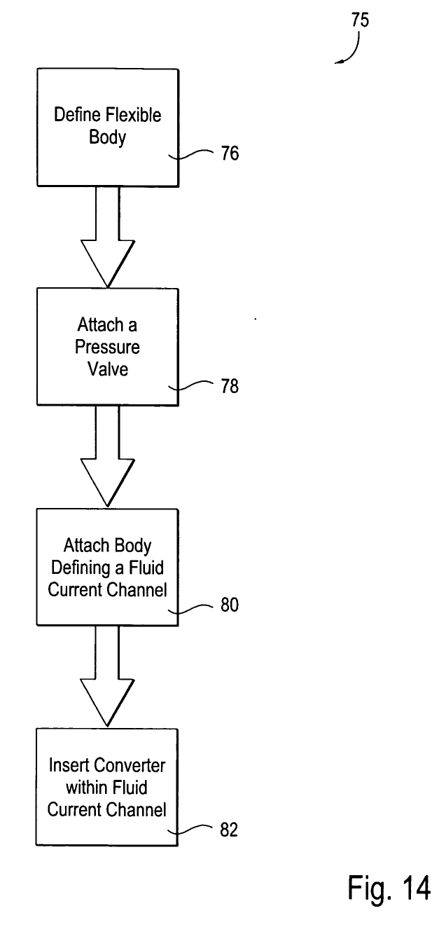

FIG. 14 shows an alternate method 75 of manufacture. In this alternate method 75, the first operation 76 is to define a flexible body. The flexible body is designed such that a fluid-filled chamber is encompassed within the flexible body and a single opening is the only way for fluid to either enter or escape this chamber. The flexible body can be made of any flexible material such as natural or manufactured rubber.

The next operation 78 is to attach a pressure valve to the single opening in the flexible body. The pressure valve functions such that in response to changes in pressure the valve can open to allow fluid to enter or exit the fluid chamber. On the other side of the pressure valve a second body is attached defining a fluid current channel, operation 80. This body is structured such that when the pressure valve opens in either direction the fluid entering or leaving the fluid chamber flows through the fluid current channel.

The next operation 82 in manufacturing is to insert a converter within the fluid current channel such that in response to fluid flows either entering or leaving the fluid chamber the converter is capable of converting the fluid flows into electronic signals. In addition, any other types of sensors, such as a pressure sensor, can be utilized instead of the converter.

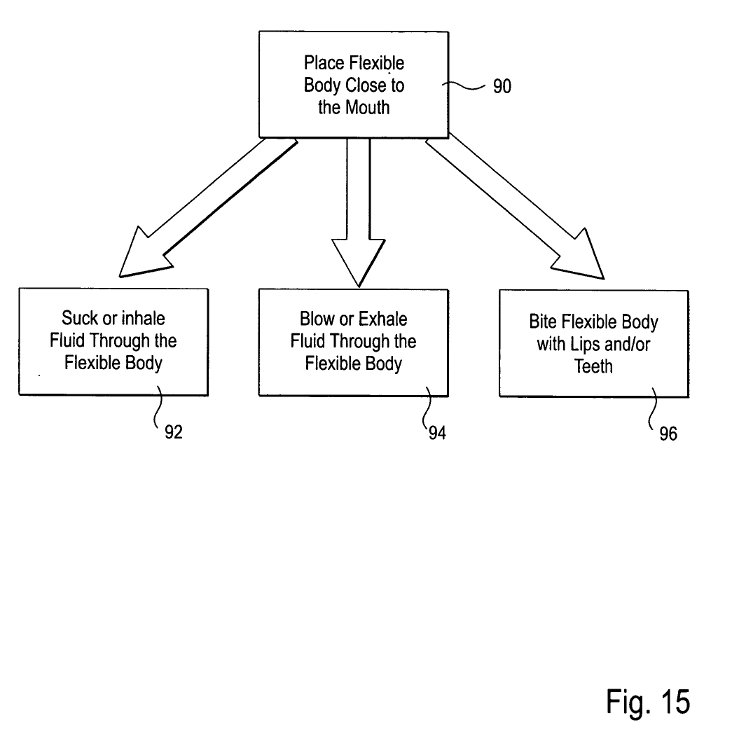

FIG. 15 shows three methods 92, 94, and 96 of using the input device. In one method, the first operation 90 is to place the flexible body close to the mouth, preferably within three inches of the mouth. The next operation 92 is to suck or inhale fluid through the flexible body. Another method of using the input device is to first place the flexible body close to the mouth 90, and then blow or exhale fluid through the flexible body 94. In a third method of using the input device, the first operation 90 is to place flexible body close to the month, while the second operation 96 is to bite the flexible body with one or both of lips and teeth. Depending on the embodiment of the input device, the method of use could be any one of the methods described above and depicted in FIG. 15 as well as a combination of two or more of them.

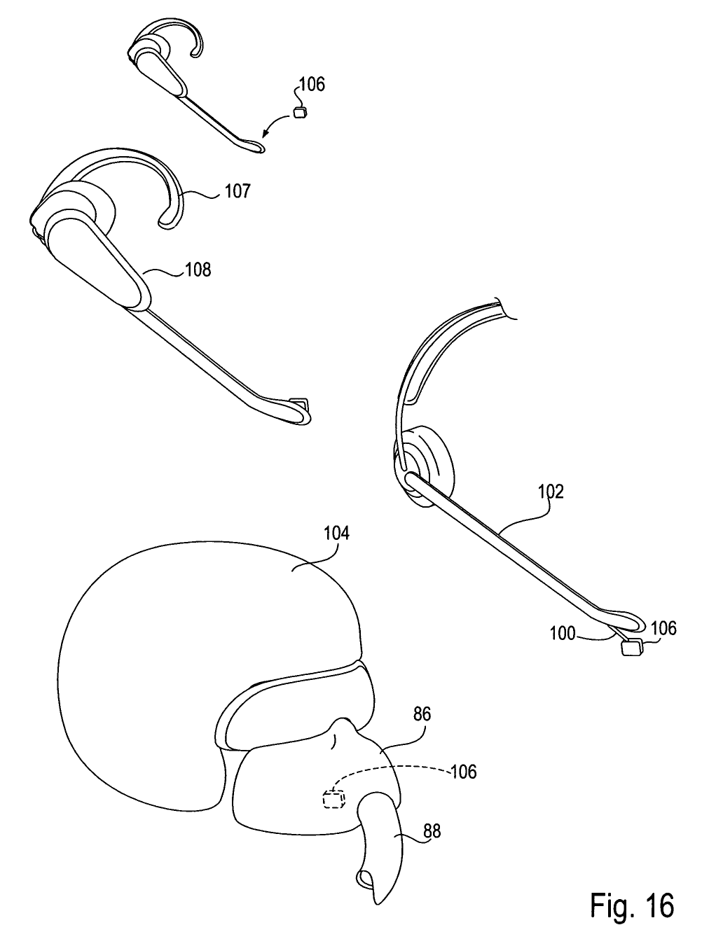

FIG. 16 displays several configurations where the input device is coupled to objects for the sake of mounting the input device near the mouth of the user and enabling hands-free use. For example, in one embodiment a headset 108 that is fitted over the ear with an ear piece 107 can be manufactured such that the input device 106 is embedded within the portion of the headset that extends in front of the user's mouth. A second embodiment shows the input device 106 attached to a headset 102 with a clip, pin, or other attachment 100. In an alternate embodiment, the input device 106 can be embedded within a helmet 104 such as those commonly used in cockpits of airplanes, other avionics systems, military and security systems. In this embodiment, the input device 106 can be embedded in the portion of the helmet that surrounds the mouth 86. In an alternate embodiment, secondary pressurized air circuits can be attached via a fluid hose 88 to the input device 106 or the portion of the helmet 104 that covers the mouth 86.

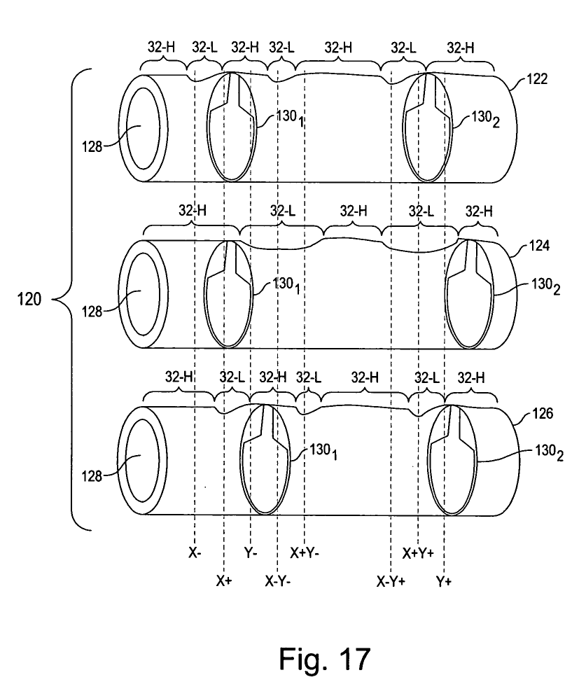

FIG. 17 illustrates one example of an application of the invention for the input device, denoted in its entirety by reference numeral 120, controlled by the bite of a user for controlling the movement of a cursor or pointer on a display screen. The exemplary input device 120 comprises three tubes 122, 124, 126, which are aligned together. Each tube 122, 124, 126 has an orifice 128 at the level that an individual can breathe out (expel) or take in (inhale) air.

Each tube comprises at least one region of relatively lesser stiffness 32-L and at least one region of relatively greater stiffness 32-H. The structure of each tube enables it to be partly distorted under the influence of a deformation force, such as biting by one or both of lips and teeth of a user. The succession of 32-L and 32-H regions enables the user to feel the different attainable features by moving lips or teeth on the input device 120. It will be appreciated by one skilled in the art that the region of relatively lesser stiffness can be made identifiable by many other techniques, structures or features, for example, lesser stiffness regions 31-L may be concave areas on the tube and the surface of greater stiffness regions 32-H may be hatch.

Each tube 122, 124, 126 has two deflection segments, 130₁ and 130₂. The deflection segments are capable of motion when the tube is distorted by bite, or by an expiration flow or inspiration flow of fluid, such as the breath of a user. The segments can deflect in one or more directions in designs where this would be needed. In this exemplary embodiment, a total of six segments are embedded in the tubes.

When the user wishes to control movement of the cursor on the display screen, the user bites on one portion of the input device 120. There is no input if the user bites on the rigid portion 32-L of the tubes. The tubes are not distorted and the segments not deflected.

To control cursor movement in a X−Y direction, that is X−Y−, X+Y−, X−Y+or X+Y+, the user has to bite a portion at which two tubes are non-rigid, hence causing a deformation of the tubes and also causing two segments to be deflected. In FIG. 17, deflection of segments 130, of tubes 122 and 124 will result in a X−Y− movement.

For linear direction movement, that is X+, X−, Y+ or Y−, only one tube 122, 124, or 126 is distorted, hence, only one segment is deflected.

Table 1 summarizes how the movement of the cursor is associated with the deflection of segments in the tubes. For example, to control cursor movement in X−Y− direction, segment 130, of each of the tubes 122 and 124 is deflected, while segment 130₂ of each of the tubes 122 and 124, segments 130, and 130₂ of tube 126 are closed. Cursor movement in Y+ direction can be achieved by deflecting segment 130₂ of tube 124.

TABLE 1
Cursor movement controlled by the deflection of segments in
each tube caused by the influence of a deformation force.

| | Cursor movement in X-Y directions | | | | | | | |
|---|---|---|---|---|---|---|---|---|
| Tube | X− | X+ | Y− | X−Y− | X+Y− | X−Y+ | X+Y+ | Y+ |
| 122 | 130₁ | — | — | 130₁ | — | 130₂ | — | — |
| 124 | — | — | 130₁ | 130₁ | 130₁ | 130₂ | 130₂ | 130₂ |
| 126 | — | 130₁ | — | — | 130₁ | — | 130₂ | — |

In another embodiment, the input device 120 can be controlled, by an expiration flow or inspiration flow, to thereby control the movement of a cursor or pointer on a display screen. The deflection segments 130₁ and 130₂ are capable of motion when the tube is distorted by an expiration or inspiration flow of fluid, such as the breath of a user.

Segment 130₁ of tubes 122 and 126 can deflect in both directions, in response to the expiration flow or inspiration flow of fluid. The segments are designed to be fluid tight, so that the flow of fluid cannot find any exhaust other than by deflecting the segments.

Segment 130₂ of each of the tubes 122 and 126 is designed so that the fluid flow will not deflect the segments. It will be appreciated by one skilled in the art that it is possible to create this region by many methods, for example, the segments are not airtight (or contain exhausts), thus allowing the fluid flow to go through the tube.

Segment 130₁ of tube 124 can only be stressed by inspiration flow of fluid and segment 130₂ expiration flow of fluid.

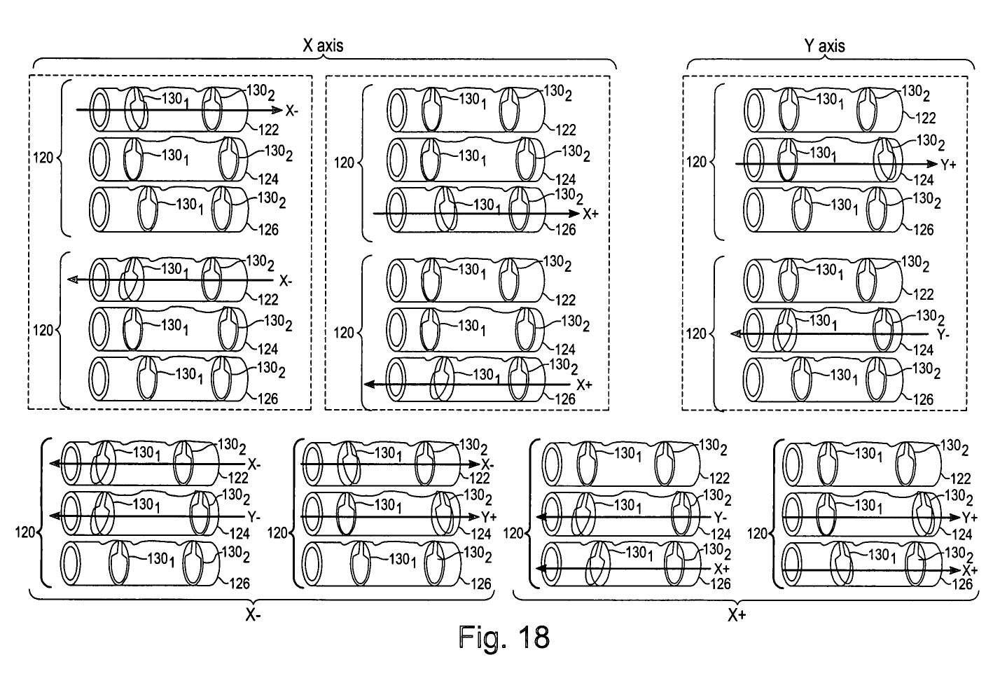

The deflection of segments in the tube stressed by the inspiration or expiration flow of fluid can be used to control cursor movement in a X−Y direction as illustrated in FIG. 18.

Table 2 summarizes how the movement of the cursor is associated with the deflection of segments in the tubes. For example, to control cursor movement in X−Y− direction, segment 130₁ of each of the tubes 122 and 124 is deflected, while segment 130₂ of each of the tubes 122 and 124, and segments 130₁ and 130₂ of tube 126 are closed. Cursor movement in Y+ direction can be achieved by deflecting segment 130₂ of tube 124.

TABLE 2
Cursor movement controlled by the deflection of segments in each
tube caused by the expiration or inspiration flow of fluid.

| | Cursor movement in X-Y directions | | | | | | | |
|---|---|---|---|---|---|---|---|---|
| Tube | X− | X+ | Y− | X−Y− | X+Y− | X−Y+ | X+Y+ | Y+ |
| 122 | 130₁ | — | — | 130₁ | — | 130₁ | — | — |
| 124 | — | — | 130₁ | 130₁ | 130₁ | 130₂ | 130₂ | 130₂ |
| 126 | — | 130₁ | — | — | 130₁ | — | 130₁ | — |

The movement speed of the cursor could depend on the intensity of the biting or blowing applied.

As to clicking, the invention incorporates the features disclosed in U.S. Provisional Patent Application No. 60/378,561, filed Jun. 05, 2002. In addition, the Boolean function can be achieved by time and/or intensity-based outputs.

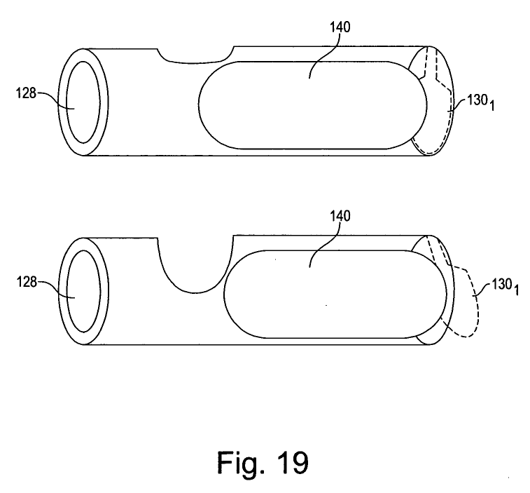

Despite the fact that the embodiments disclosed above illustrate the functions that are ordinarily performed with the use of a mouse in a GUI environment, the present invention's principle is not restricted to a two-dimensional environment. The present invention is applicable to three and higher dimensional pointing and tracking (by repeating the present means in several planes and using several combinations of pointing and clicking devices), to scroll function as well, and can be used in a variety of fields, like games, ergonomic controls, etc. In one embodiment, the new device provides the user with different ways of controlling a computer or an electronic system through a single organ (e.g., a mouth), a single action (bite or inflow of fluid) or dual actions (combination of bite and inflow of fluid), thus not interfering with other organs (e.g., hands or eyes), which could be used for other input or output purposes. In another embodiment, the user can alternate between bite and breath to achieve the same control functionalities, depending on the preferred means of interaction for the user. One skilled in the art will find other designs in line with the invention. In one exemplary design, a mobile element 140 can come to push against the segments, as illustrated in FIG. 19. It should be noted that this device may have various shapes and may be embedded or integrated in a variety of portable, wearable devices.

In one example of an application, the present invention may be utilized for controlling a variety of functions inside the general aviation (commercial and military) and aerospace's so-called “cockpits of the future”. In one embodiment, mechanical or electromechanical device may generate the airflow. It should be noted that the present invention is not limited to an airflow, and may operate using any gaseous or liquid flow. The fluid flow may be generated by the cooling systems (generally based on adjustable air circulation to prevent overheating in the cockpit), by compressors that supply gas to the pneumatic systems used for inflation/deflation of anti-G suits, pressurized circuits, the oxygen mask circuit or any air, approved gas, or even liquid flow source with suitable output and pressure. The invention enables pilot to control and command various functions through cockpit's GUIs when conditions do not allow normal breathing, such as when the aircraft is accelerating.

Thus a method and device for providing input to a computer system have been described. Although the present invention has been described with reference to specific exemplary embodiments, it will be evident that various modifications and changes may be made to these embodiments without departing from the broader spirit and scope of the invention. Accordingly, the specification and drawings are to be regarded in an illustrative rather than a restrictive sense.

1. A device to provide input to a computer system, the device including:
a flexible body defining a fluid current channel having an inlet and an outlet;
a region of relatively greater stiffness within the flexible body defining the fluid current channel;
a lever segment, anchored to the region of relatively greater stiffness so as to be substantially parallel to a wall of the fluid current channel; and
a displaceable member that depends from the flexible body and is anchored to the region of relatively greater stiffness, the displaceable member being movable within the flexible body to generate an electric signal.

2. The device of claim 1 including:
a converter to convert motion of the displaceable member into the electric signal;
a processor to process the electric signal generated by the converter; and
an interface between the processor and the electronic or computer system.

3. The device of claim 1, wherein the flexible body that defines the fluid current channel is a tube.

4. The device of claim 1, wherein the region of relatively greater stiffness is a ring embedded in the fluid current channel.

5. The device of claim 1, wherein the displaceable member is movable in response to a fluid current flowing through the fluid current channel.

6. The device of claim 5, wherein the fluid current is an exhaled breath.

7. The device of claim 5, wherein the fluid current is an inhaled breath.

8. The device of claim 5, wherein the fluid current is in one or both of gaseous and liquid states.

9. The device of claim 1, wherein the displaceable member is movable in response to deformation of the flexible body defining the fluid current channel.

10. The device of claim 9 wherein the deformation of the flexible body defining the fluid current channel is caused by biting with one or both of lips and teeth.

11. The device of claim 1 wherein the computer system is an avionics system.

12. The device of claim I including at least two regions of relatively greater stiffness, wherein at least one substantially parallel lever segment and at least one displaceable member are anchored to each of the at least two regions of relatively greater stiffness.

13. The device of claim 1, wherein the displaceable member is a deflection segment.

14. A device to provide input to a computer system, the input device including:
a flexible body defining a chamber with an opening, the chamber to accommodate a fluid;
a pressure valve attached to the opening that is capable of opening and closing in response to one or both of flexible body deformation and fluid flow;
a channel body defining a fluid current channel that is in fluid communication with the chamber via the pressure valve;
a converter within the fluid current channel to convert fluid flow within the fluid current channel into an electrical signal; and
an interface between the converter and the computer system;

15. The device of claim 14 wherein the flexible body is deformable by biting by one or both of lips and teeth.

16. The device of claim 14 wherein the flexible chamber contains a spring to return the flexible chamber to an inflated condition after deformation.

17. The device of claim 14 attached to a flexible mounting rod.

18. The device of claim 14 attached to a headset.

19. The device of claim 14 attached to at least one circuit of pressurized fluid.

20. A method of manufacturing a device to provide input to a computer system including:
defining a flexible body around a fluid current channel having an inlet and an outlet;
defining at least one region of relatively greater stiffness for the flexible body; and
anchoring a lever segment to the flexible body at the region of relatively greater stiffness; and
anchoring a displaceable member to the flexible body at the region of relatively greater stiffness.

21. The method of claim 20 wherein the defining of the region of relatively greater stiffness includes utilizing a material of relatively greater stiffness in place of a material that defines the flexible body outside the region of relatively greater stiffness.

22. The method of claim 20 wherein the defining of the region of relatively greater stiffness includes inserting a supporting frame within the fluid current channel.

23. The method of claim 20 wherein the defining of the region of relatively greater stiffness includes increasing the thickness of the flexible body in the region of relatively greater stiffness.

24. A method of manufacturing a device to provide input to a computer system, including:
forming a flexible body to define a chamber encasing one or more fluids and having an opening;
attaching a pressure valve to the opening;
attaching a body defining a fluid current channel to the opening such that the pressure valve forms the inlet to the fluid current channel; and
inserting a converter within the fluid current channel to convert fluid flow within the fluid current channel into an electrical signal;

25. A method of providing input to a computer system including:
placing a flexible body, defining a fluid current channel having an inlet and outlet, close to the mouth; and
creating a fluid flow within the fluid current channel by blowing a fluid into the flexible body with the mouth.

26. A method of providing input to a computer system including:
placing a flexible body, defining a fluid current channel having an inlet and outlet, close to the mouth; and
creating a fluid flow within the fluid current channel by sucking a fluid through the flexible body with the mouth.

27. A method of providing input to a computer system including:
placing a flexible body, defining a fluid current channel having an inlet and outlet, close to the mouth; and
deforming the flexible body by biting the flexible body with one or both of the teeth and lips.

28. A method of providing input to a computer system including:
placing a flexible body defining a fluid chamber close to the mouth; and
deforming the flexible body by biting the flexible body with one or both of teeth and lips.

29. A method for controlling movement of a cursor in two directions on a computer system, the method including:
detecting deflection of first and second segment, each of the first and second segments being located in one of a plurality of housings, the deflections of the first and second segments being caused by a biting action on the tube;
converting the deflections of the first and second segments into first and second signals; and
processing the first and second signals utilizing a processor, so that the deflections of the first and second segments correspond to a direction of movement of the cursor.

30. An apparatus for controlling movement of cursor in two directions on a computer screen, the apparatus comprising:
means for deforming a flexible body of a plurality of housings;
means for deflecting first and second segments, each of the first and second segments being located in one of the plurality of housings;
means for detecting the deflections of the first and second segments;
means for converting the deflections of the first and second segment into first and second signals; and
means for processing the first and second signals to correspond to a direction of movement of the cursor.

31. The apparatus of claim 30, wherein the means for deforming the flexible body of the plurality of housings includes each of the plurality of housings constructed to have a region of relatively lesser stiffness and a region of relatively greater stiffness.

32. The apparatus of claim 30, wherein the means for deflecting the first and second segments includes a biting action on the housings, each of the first and second segments being anchored to the region of relatively greater stiffness.

33. The apparatus of claim 30, wherein the means for deflecting the first and second segments includes at least a mobile element within each of the plurality of housings, the mobile element pushing against at least one of the first and second segments.

34. The apparatus of claim 30, wherein the means for detecting the deflections of the first and second segments includes a concaved region of lesser stiffness and a hatch region of relatively great stiffness is hatch.

35. The apparatus of claim 30, wherein the means for converting the deflections of the first and second segments into first and second signals includes a converter.

36. The apparatus of claim 30, wherein the means for processing the first and second signals to corresponds to a direction of movement of the cursor is a processor.
